(19) United States
(12) Patent Application Publication
(10) Pub. No.: US 2022/0044766 A1
(43) Pub. Date: Feb. 10, 2022

(54) CLASS-DEPENDENT MACHINE LEARNING BASED INFERENCES

(71) Applicant: International Business Machines Corporation, Armonk, NY (US)

(72) Inventors: Alessandra Toniato, Thalwil (CH); Philippe Schwaller, Duedingen (CH); Teodoro Laino, Rueschlikon (CH)

(21) Appl. No.: 16/984,331

(22) Filed: Aug. 4, 2020

(51) Int. Cl. G16C 20/10; G06N 3/04; G06N 5/04; G06K 9/62; G06F 40/284

(52) U.S. Cl. CPC G16C 20/10; G06N 3/04; G06F 40/284; G06K 9/6256; G06K 9/6228; G06N 5/04

(57) ABSTRACT

A computer-implemented method of performing class-dependent, machine learning based inferences includes accessing a test input and N class identifiers, wherein each class identifier of the N class identifiers identifies a respective class among M possible classes; forming N test input data structures, wherein each test input data structure of the N test input data structures is formed by aggregating the test input with a different one of the N class identifiers; performing an inference for each of the N test input data structures using a machine learning model that is trained using examples associating example input data structures with respective example outputs, wherein each respective example input data structure is formed by aggregating an example input with a different one of the N class identifiers; and returning a class-dependent inference result for each respective test input data structure based on the inference obtained for each respective test input data structure.

### BACKGROUND

The present invention generally relates to computer-implemented techniques for performing machine learning based inferences, and more specifically, to a computer-implemented method, computer system and computer program product for performing class-dependent machine learning based inferences associated with chemical retrosynthetic analysis.

Machine learning often relies on artificial neural networks (ANNs), which are computational models inspired by biological neural networks in human or animal brains. Such systems progressively and autonomously learn tasks by means of examples and have successfully been applied to speech recognition, text processing, and computer vision. Typically, an ANN includes a set of connected units or nodes, which can be likened to biological neurons and are therefore referred to as artificial neurons. Signals are transmitted along connections (also called edges) between artificial neurons, similar to synapses. That is, an artificial neuron that receives a signal processes it and then signals other connected neurons. Many types of neural networks are known, including feedforward neural networks, such as multilayer perceptrons, deep neural networks, and convolutional neural networks. Sophisticated network architectures have been proposed, notably in the fields of natural language processing, language modeling, and machine translation, see, e.g., “Attention Is All You Need”, Ashish Vaswani et al., in Advances in Neural Information Processing Systems, pages 6000-6010.

Neural networks are typically implemented in software. However, a neural network may also be implemented in hardware, for example, as a resistive processing unit or an optical neuromorphic system. Machine learning can notably be used to control industrial processes and make decisions in industrial contexts. Amongst many other examples, machine learning techniques can also be applied to retrosynthetic analyses, which are techniques for solving problems in the planning of organic syntheses. Such techniques aim to transform a target molecule into simpler precursor structures. The procedure is recursively implemented until sufficiently simple or adequate structures are reached.

### SUMMARY

According to one embodiment of the present invention, a computer-implemented method of performing class-dependent, machine learning based inferences is disclosed. The computer implemented method includes accessing a test input and N class identifiers, wherein each class identifier of the N class identifiers identifies a respective class among M possible classes. The computer-implemented method further includes forming N test input data structures, wherein each test input data structure of the N test input data structures is formed by aggregating the test input with a different one of the N class identifiers. The computer-implemented method further includes generating an inference for each of the N test input data structures using a machine learning model that is trained using examples associating example input data structures with respective example outputs, wherein each respective example input data structure is formed by aggregating an example input with a different one of the N class identifiers. The computer-implemented method further includes returning a class-dependent inference result for each respective test input data structure based on the inference generated for each respective test input data structure.

According to another embodiment of the present invention, a computer-implemented method of machine learning based retrosynthesis planning is disclosed. The computer-implemented method includes accessing a test input and N class identifiers, wherein the test input is a string specifying a structure of a chemical species corresponding to a chemical reaction product, and each class identifier of the N class identifiers is a string identifying a respective class among M possible classes of chemical reactions. The computer-implemented method further includes forming N test input data structures, wherein each test input data structure of the N test input data structures is formed by concatenating the test input with a different one of the N class identifiers. The computer-implemented method further includes generating an inference for each of the N test input data structures using a machine learning model that is trained using examples associating example input data structures with respective example outputs, wherein each respective example input data structure is formed by concatenating an example input with a different one of the N class identifiers, each respective input data structure is a string specifying structures of chemical species corresponding to chemical reaction products, and each respective example output is a string formed by aggregating specifications of structures of two or more precursors of the chemical reaction products. The computer-implemented method further includes returning a class-dependent inference result for each respective test input data structure based on the inference generated for each respective test input data structure.

According to another embodiment of the present invention, a computer system for performing class-dependent, machine learning based inferences is disclosed. The computer system includes one or more computer processors, one or more computer readable storage media, and program instructions stored on the computer readable storage media for execution by at least one of the one or more processors. The program instructions include instructions to access a test input and N class identifiers, wherein each class identifier of the N class identifiers identifies a respective class among M possible classes. The program instruction further include instructions to form N test input data structures, wherein each test input data structure of the N test input data structures is formed by aggregating the test input with a different one of the N class identifiers. The program instructions further include instructions to generate an inference for each of the N test input data structures using a machine learning model that is trained using examples associating example input data structures with respective example outputs, wherein each respective example input data structure is formed by aggregating an example input with a different one of the N class identifiers. The program instructions further include instructions to return a class-dependent inference result for each respective test input data structure based on the inference generated for each respective test input data structure.

### BRIEF DESCRIPTION OF THE DRAWINGS

The drawings included in the present application are incorporated into, and form part of, the specification. They illustrate embodiments of the present invention and, along with the description, serve to explain the principles of the present invention. The drawings are only illustrative of certain embodiments and do not limit the present invention. The same reference numbers used throughout the drawings, unless otherwise indicated, shall generally refer to the same components in the various embodiments of the present invention.

FIG. 1 depicts a cloud computing environment in accordance with at least one embodiment of the present invention.

FIG. 2 depicts abstraction model layers in accordance with at least one embodiment of the present invention.

FIG. 3 depicts a flowchart diagram of a method of performing class-dependent, machine learning based inferences in accordance with at least one embodiment of the present invention.

FIG. 4 depicts a flowchart diagram of a training method to obtain a cognitive model for generating class-dependent inferences in accordance with at least one embodiment of the present invention.

FIG. 5 depicts a flowchart diagram of a method for preparing a training set of suitable examples for training a machine learning model that can subsequently be used to perform class-dependent inferences in accordance with at least one embodiment of the present invention.

FIGS. 6A-6G is a sequence depicting steps for preparing an example associating a given input (a chemical reaction product) to a given output (a set of precursors for that product) in accordance with at least one embodiment of the present invention. Here, an an input data structure is formed by aggregating the given input with a class identifier identifying an automatically detected type of chemical reaction. The input data structure is then tokenized (the output is similarly processed) in view of training a machine learning model.

FIG. 6A depicts an exemplary chemical reaction, including a given chemical product (input) and given precursors (output) in accordance with at least one embodiment of the present invention.

FIG. 6B depicts SMILE system string representations of the input and output of FIG. 6A in accordance with at least one embodiment of the present invention.

FIG. 6C depicts a functional group interconversion corresponding to class identifier 9 derived from classifying the SMILE system string representations of FIG. 6B.

FIG. 6D depicts an input data structure formed by aggregating the functional group interconversion corresponding to class identifier 9 of FIG. 6C with the inputs of FIG. 6B.

FIG. 6E depicts an example datum formed from the input data structure of FIG. 6D and the output of FIG. 6A in accordance with at least one embodiment of the present invention.

FIG. 6F depicts splitting of the input data structure of FIG. 6D into tokens in accordance with at least embodiment of the present invention.

FIG. 6G depicts the tokens formed from the input data structure of FIG. 6D as a result of tokenization of the input data structure in accordance with at least one embodiment of the present invention.

FIGS. 7A-7C is a sequence depicting steps for using tokens extracted from an input data structure to obtain embeddings (i.e., extracted vectors), in which the embeddings are fed into a suitably trained model to perform class-dependent inferences in accordance with at least one embodiment of the present invention.

FIG. 7A depicts the tokens of FIG. 6G in accordance with at least embodiment of the present invention.

FIG. 7B depicts an exemplary embedding of the tokens of FIG. 7A in accordance with at least one embodiment of the present invention.

FIG. 7C depicts an exemplary machine learning model having an encoder-decoder structure for performing inferences for an input data structure in accordance with at least one embodiment of the present invention.

FIG. 8 depicts a cloud computing node in accordance with at least one embodiment of the present invention.

### DETAILED DESCRIPTION

Machine learning models are typically trained using data collected from proprietary or public datasets. Unfortunately, when specific data regions are poorly represented, statistically speaking, inferences performed with the resulting cognitive model will be impacted by limited confidence in predictions corresponding to such regions.

Typically, the “most effective” solutions provided by a cognitive model will rank high in terms of accuracy since the inference confidence is effectively biased by the amount of similar data seen during the training. Therefore, solutions corresponding to training areas where a large amount of example data is available for the training will be favored, compared with solutions predicted based on areas with low data volumes. Embodiments of the present invention recognize that this can be problematic when a cognitive model is applied to industrial processes in which heterogeneously distributed training datasets are available. This stems from the fact that the true optimal solution may not necessarily be the solution with the highest confidence, but rather one that is ignored (though still predicted) because of its lower confidence. This is notably true when applying machine learning to retrosynthetic analyses. Accordingly, embodiments of the present invention recognize that it may be desirable to achieve a wider collection or range of reasonable inferences, which are not clouded by an inference confidence bias.

Embodiments of the present invention provide for an improvement to the aforementioned problems through various methods that rely on classified (or categorized) data inputs to perform class-dependent inferences. Such methods require machine learning models to be consistently trained, for example, based on example input data associating classified inputs to respective outputs. Advantageously, this approach can reuse existing machine learning network architectures, provided that the training datasets are suitably modified.

According to various embodiments of the present invention, class-dependent, machine learning based inferences are performed. A test input and N class identifiers are accessed. Each class identifier identifies a respective class among M possible classes. N test input data structures are formed from the test input by combining the test input with a different one of the N class identifiers. Inferences are performed for each of the test input data structures using a cognitive model obtained by training a machine learning model based on suitably prepared examples. Such examples associate example input data structures with respective example outputs, wherein the example input data structures are formed by combining an example with a different one of the N class identifiers. Class-dependent inferences results obtained with regards to the test input are returned based on the inferences performed for each of the test input data structures.

In other words, embodiments of the present invention rely on classified (or categorized) data inputs to perform class-dependent inferences, for example, to make class-dependent predictions or classifications. The underlying machine learning model must be trained based on examples that are prepared in a manner that is consistent with the aggregation mechanism used for inferences. Notwithstanding, embodiments of the present invention can reuse existing machine learning network architectures and training algorithms, provided that training datasets are suitably modified. Accordingly, embodiments of the present invention can advantageously be applied to retrosynthetic analyses, computer-aided design, computer-aided engineering, or defect or failure predictions, amongst other applications, while reducing confidence bias in machine learning-based inferences.

In an embodiment, upstream training steps (i.e. training steps prior to accessing the classified test set) are used to train the model. Here, a training set is accessed, which includes examples associating example input data structures with respective example outputs. The example input data structures are formed by aggregating the example inputs with respective class identifiers. The machine learning model is trained according to such examples. Inferences are performed based on N sets of features extracted from the example input data structures, respectively. Likewise, the machine learning model used for a given classified test set is a model trained based on features extracted from the examples, including features extracted from the example input data structures.

In an embodiment, each of the N test input data structures are formed by aggregating or concatenating a string representing the test input with a string representing a different one of the N class identifiers. Likewise, each of the example data inputs used to train the machine learning model are formed by aggregating or concatenating strings representing an example input with strings representing a different one of the class identifiers.

In an embodiment, the N sets of features are extracted from tokenized versions of the N input data structures. Likewise, the machine learning model used in that case is a model trained based on features extracted from tokenized versions of the example data structures. Each of the tokenized versions is obtained by applying a same tokenization algorithm. Example outputs can similarly be processed.

In an embodiment, the machine learning model used includes an encoder-decoder structure, which includes one or more encoders connected to one or more decoders. Each of the encoders and each of the decoders include an attention layer and a feed-forward neural network, interoperating so as to perform inferences by predicting probabilities of possible outputs based on which class-dependent inference result is returned. The model, may, for instance, have a sequence-to-sequence architecture.

In an embodiment, the strings representing the test input, the example inputs, and the class identifiers are all obtained according to a same set of syntactic rules and the tokenization algorithm is devised in accordance with the set of syntactic rules. This helps to achieve more consistent and reliable outputs.

In an embodiment, the strings representing the class identifiers are obtained so as to give rise to respective tokens (or sets of tokens) upon applying the tokenization algorithm.

In an embodiment, strings representing the test input and the example inputs are ASCII strings specifying structures of chemical species corresponding to chemical reaction products. Likewise, each of the example outputs used to train the machine learning model are ASCII strings formed by aggregating respective specifications of structures of two or more precursors of such chemical reaction products. For example, the ASCII strings can be formulated according to the simplified molecular-input line-entry (SMILE) system.

In an embodiment, classes pertain to one or more of the following categories of chemical reactions: unrecognized chemical reaction, heteroatom alkylation and arylation, acylation and related processes, C—C bond formation; heterocycle formation, protection, deprotection, reduction, oxidation, functional group interconversion, functional group addition, and resolution reaction. In addition, one of the classes may pertain to unrecognized chemical reactions, so as to allow any example to be classified.

In an embodiment, the number N of class identifiers used on inferences may be equal to M number of possible classes. In this case, inferences are performed for all of the classes available (as used for training purposes). In an embodiment, only a subset of the M number of possible classes are used on inferences (N is strictly smaller than M in this case). For example, N class identifiers are automatically selected based on the accessed test input, which may be achieved thanks to machine learning or any other suitable automatic selection method. In an embodiment, the test input and N class identifiers to be accessed is based on a user selection of the test and N class identifiers. In other words, the user specifies the classes of interest.

It should be appreciated that embodiments of the present invention can be utilized to perform retrosynthesis planning. A test input and N class identifiers are accessed, wherein the test input is a string specifying a structure of a chemical species corresponding to a chemical reaction product and each of the N class identifiers is a string identifying a respective class of chemical reactions among M possible classes, where M≥N≥2. N test input data structures are formed from the test input by concatenating the test input with a respective one of the N class identifiers, N≥2. Inferences are performed for each of the N test input data structures using a machine learning model trained according to examples associating example input data structures with respective example outputs. Each of the example input data structures are formed by concatenating the example inputs with a different one of the N class identifiers, wherein the example inputs are strings specifying structures of chemical species corresponding to chemical reaction products. Similarly, each of the example outputs are strings formed by aggregating specifications of structures of two or more precursors of the chemical reaction products. A class-dependent inference result for each respective test input data structure is returned based on an inference obtained for each respective test input data structure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suit-able combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

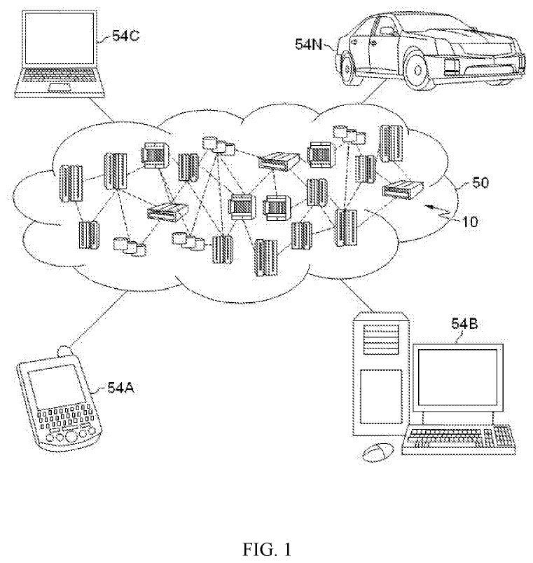

Referring now to FIG. 1, a cloud computing environment in accordance with at least one embodiment of the present invention is depicted. Cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Cloud computing nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 1 are intended to be illustrative only and that cloud computing nodes 10 and cloud computing environment 50 can communicate with any type of computerized device over any type of network and/or network addressable connection (e.g., using a web browser).

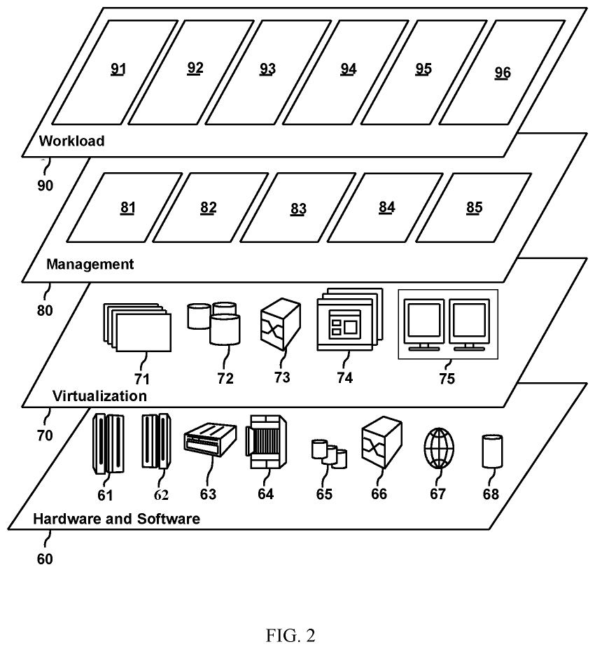

Referring now to FIG. 2, a set of functional abstraction layers provided by cloud computing environment 50 (shown in FIG. 1) is depicted. It should be understood in advance that the components, layers, and functions shown in FIG. 2 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and class-dependent machine learning based inferences 96.

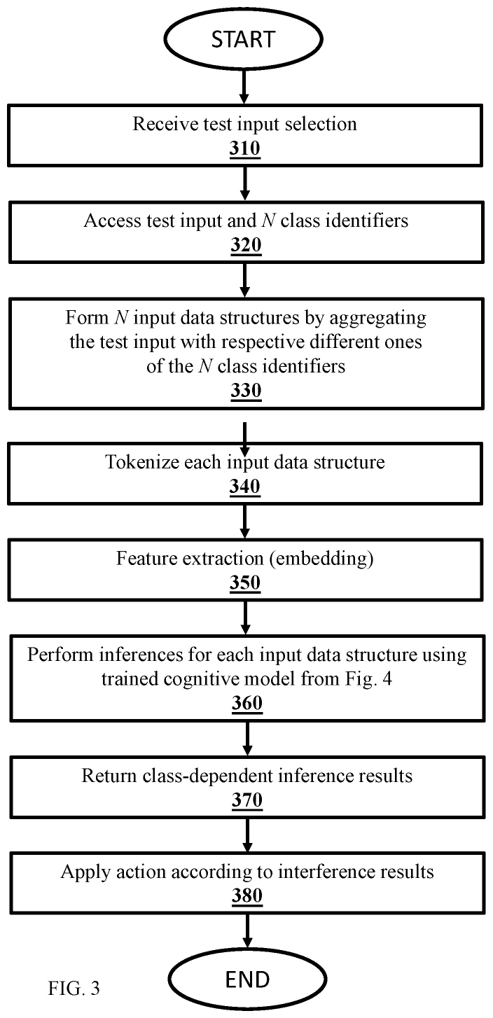

Referring now to FIG. 3, a flowchart diagram of a method of performing class-dependent, machine learning inferences in accordance with at least one embodiment of the present invention is depicted. At step 310, a test input and N class identifiers are accessed. Each identifier identifies a respective class among M classes. In an embodiment, M≥N≥2. In an embodiment, N=M. In an embodiment, N<M. Typically, the test input includes information associated with a target, for which responses are needed. The class identifiers are used to categorize outputs to be returned according to given classes in accordance with embodiments of the present invention. The M classes may generally pertain to inputs, outputs, or to a relation between such inputs and outputs. For example, such classes may concern different types of chemical reactions, whereas the inputs and outputs may respectively relate to chemical reaction products and precursors of such products. In this case, the class identifiers are used to categorize sets of precursors of products, according to different possible types of chemical reactions.

At step 320, N test input data structures are formed, wherein each of the resulting test input data structures are formed by aggregating the test input with a different one of the N class identifiers. That is, a single test input eventually gives to N data structures that will be fed as inputs to a cognitive model. For example, each of the test input and the class identifiers may be strings that are aggregated or concatenated in step 330.

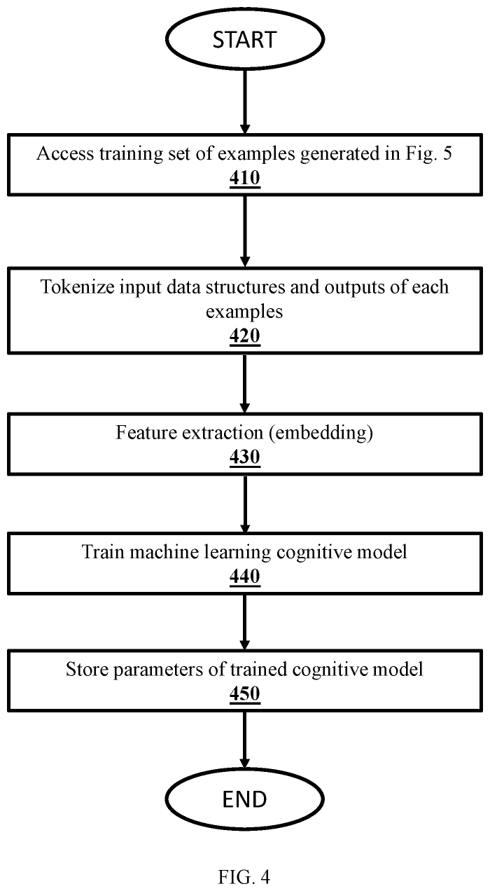

In an embodiment, each of the N test input data structures are formed by concatenating a string representing the test input with a string representing a different one of the N identifiers. Similarly, each of the example input data structures used to train the cognitive model in accordance with FIG. 4 are formed by concatenating strings representing the example inputs with strings representing respective ones of the class identifiers.

One of ordinary skill in the art will appreciate that using strings allows for translation engines to be leveraged to obtain internal representations of the inputs, which are then translated into the most probable outputs, while also taking into account the context in which “words” appear in the input. However, in embodiments of the present invention, tokens are preferably used, instead of words. Like words, such tokens can be regarded as small, identifiable sequences of characters of the strings. Moreover, like words, such tokens normally correspond to respective (e.g., unique) entries in a model vocabulary, which can be processed separately in the embedding step. In an embodiment, instead of tokens, the extraction may proceed character by character. However, using tokens may yield results that are more relevant, semantically speaking.

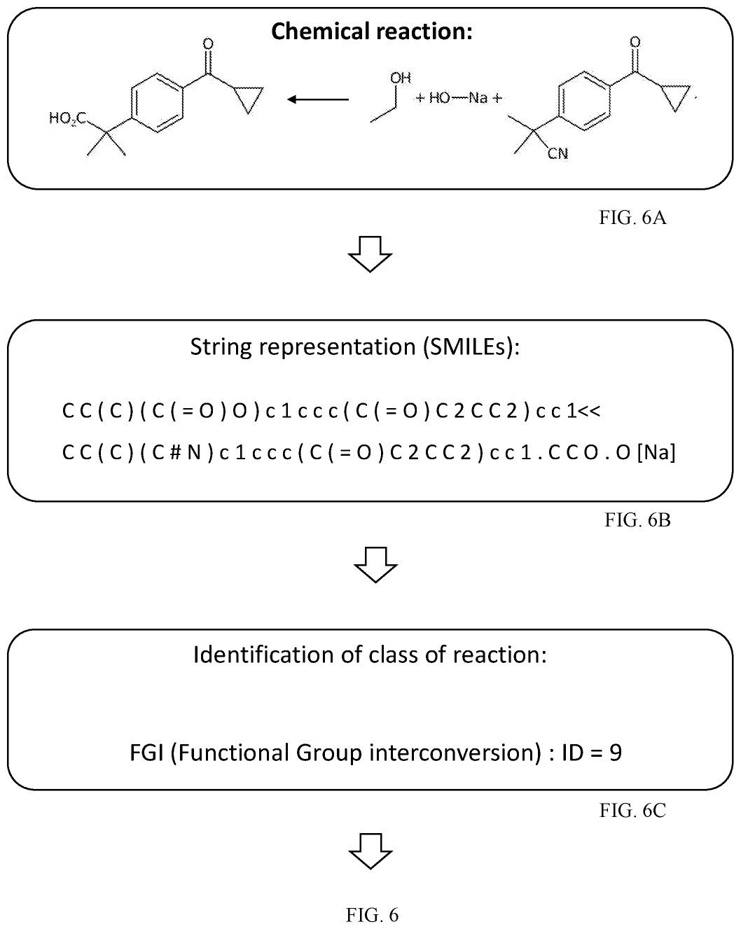

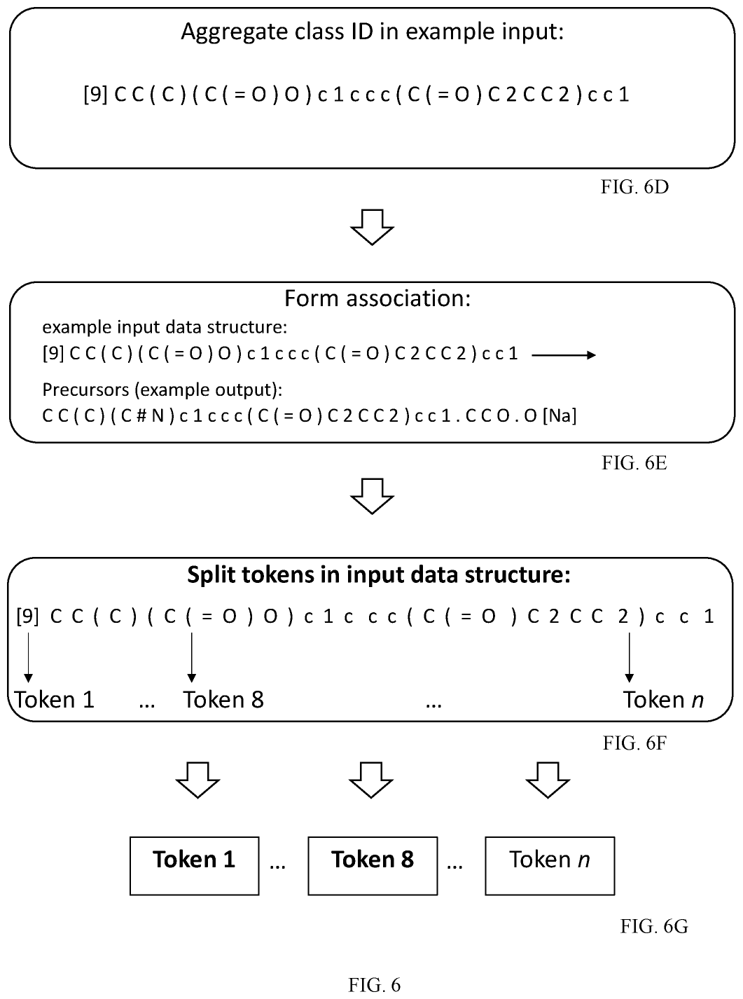

It should be noted that the strings representing the test input, the example inputs, and the class identifiers are preferably obtained according to a same set of syntactic rules. In that case, the tokenization algorithm is normally devised in accordance with the syntactic rules. In particular, the syntactic rules and the tokenization algorithm may be devised in such a manner that the strings representing the class identifiers will give rise to respective tokens upon applying the tokenization algorithm. In typical scenarios, a class identifier gives rise to a respective token (e.g., Token 1 in FIG. 6B), while the rest of the input data structure (corresponding to the initial input) may give rise to several tokens (e.g., Tokens 2 to n in FIG. 6B).

At step 340, each of the test input data structures are tokenized in view of a feature extraction (embedding) step to be performed at step 350. At step 350, N set of features are extracted from each of the tokenized versions of the test input data structures. It should be noted that each token may give rise to a respective vector (e.g., as assumed in FIG. 6B). Thus, a given test input gives rise to N test input data structures, each of which leads to L tokens, L being the number of tokens extracted from each of the N test input data structures. Thus, each of the N sets of extracted features may actually involve L vectors, as further assumed in FIG. 6B, which schematically depicts vectors obtained from a single test input data structure.

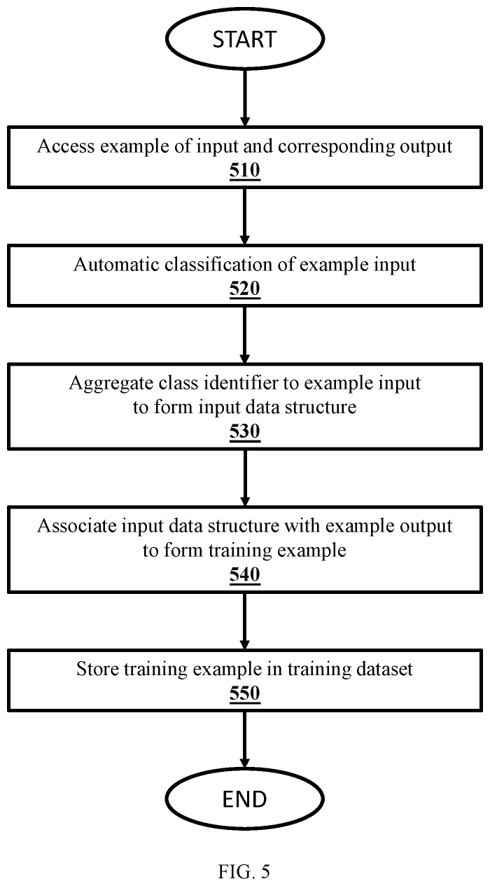

At step 360, inferences are performed for each of the test input data structures using a suitable cognitive model (e.g., the cognitive model generated prepared in accordance with FIG. 4). The cognitive model is a machine learning model that has been trained according to suitably prepared examples (e.g., the training set of suitable examples prepared in accordance with FIG. 5). The examples associate example input data structures with respective example outputs. Consistent with the test input data structures, each of the example input data structures aggregates an example input with a respective different one of the class identifiers, wherein each class identifier identifies a respective one of the M classes. It should be noted that some pre-processing may be involved, e.g., to tokenize the input data structures and outputs, as illustrated later in reference to FIGS. 6A-6G.

At step 370, class-dependent inference results for each respective test input data structure are returned based on the inference obtained (at step 360) for each respective test input data structure. It should be noted that the results obtained may need to be sorted according to corresponding class identifiers. However, the test outputs obtained may already be sorted, by construction.

At step 380, a user may use the results, for example, to react precursors to obtain the target product according to a given type of chemical reaction as later discussed below.

It should be appreciated that during the inference phase (step 360), a test input is systematically aggregated with class identifiers (e.g., some or all of the available class identifiers) so as to allow class-dependent inferences to be performed that are consistent (in statistical terms) with the examples used for training purposes. Accordingly, results can be obtained for certain classes (or all of them), which would else be ignored by a conventional inference mechanism, owing to the confidence bias discussed earlier.

In other words, embodiments of the present invention rely on classified (or categorized) data inputs to perform class-dependent inferences, e.g., make class-dependent predictions or classifications. The inference and training mechanisms rely on consistently prepared input data structures, which integrate class identifiers. It should be appreciated that embodiments of the present invention can advantageously reuse existing machine learning network architectures, provided that training datasets are suitably modified to incorporate class identifiers.

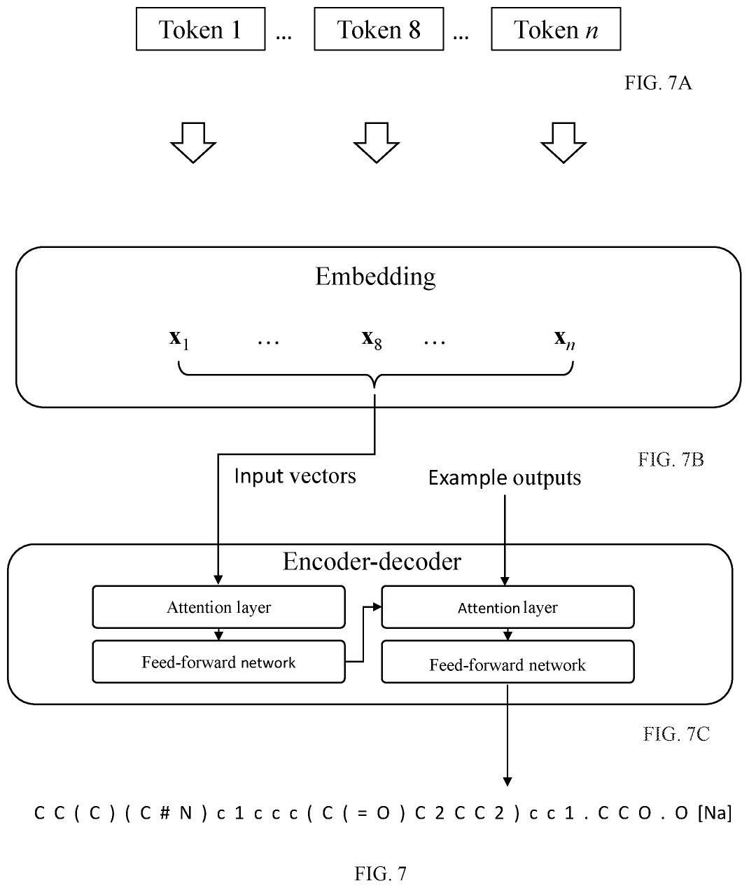

In applications to retrosynthesis planning, the strings representing the test input and the example inputs may be, for example, ASCII strings specifying structures of chemical species corresponding to chemical reaction products. Similarly, the example outputs used to train the machine learning model (e.g., in accordance with FIG. 4) may also be ASCII strings, each of which are formed by aggregating specifications of structures of two or more precursors of chemical reaction products. For example, such ASCII strings can be formulated according to the SMILE system (SMILEs), as assumed in FIGS. 6-7.

Referring again to FIG. 3, the flowchart diagram of the method of performing class-dependent, machine learning inferences will be used in the context of retrosynthesis planning in accordance with at least one embodiment of the present invention. At step 320, a test input and N class identifiers are accessed. Here, the test input is a string specifying a structure of a chemical species corresponding to a chemical reaction product and each of the N class identifiers is a string identifying a respective class of chemical reactions among M possible classes. In an embodiment, M≥N≥2. In an embodiment, N=M. In an embodiment, N<M.

At step 330, N test input data structures are formed, wherein each of the resulting test input data structures are formed by concatenating the test input with a respective different one of the N class identifiers, where N≥2.

At step 340, each of the test input data structures are tokenized in view of a feature extraction (embedding) step to be performed at step 350. At step 350, N set of features are extracted from each of the tokenized versions of the test input data structures. It should be noted that each token may give rise to a respective vector (e.g., as assumed in FIG. 7B). Thus, a given test input gives rise to N test input data structures, each of which leads to L tokens, L being the number of tokens extracted from each of the N test input data structures. Thus, each of the N sets of extracted features may actually involve L vectors, as further assumed in FIG. 7B, which schematically depicts vectors obtained from a single test input data structure.

At step 360, inferences are performed for each of the test input data structures using a suitable machine learning model (e.g., the machine learning model prepared in accordance with FIG. 4) trained according to examples associating example input data structures with respective example outputs. Each of the example input data structures are formed by concatenating example inputs with a respective different one of the N class identifiers. Here, the example inputs are strings specifying structures of chemical species corresponding to chemical reaction products and each of the example outputs are strings formed by aggregating specifications of structures of two or more precursors of the chemical reaction products.

At step 370, class-dependent inference results for each respective test input data structure are returned based on the inference obtained (at step 360) for each respective test input data structure.

Embodiments of the present invention recognize that one of the main issues of machine learning-based retrosynthesis planning algorithms is that the usual disconnection strategies lack in diversity. When the goal is to find a suitable set of precursors for a given target molecule, the generated precursors typically fall in the same chemical macro class (for example protection, deprotection) or same C—C bond formation with a slightly different set of reagents, such that the automatic synthesis planning tool invariably predicts the same results, which may not necessarily be true optimal results.

Such cognitive models preclude a broad exploration as they focus on, for example, the top single-step predictions, which differ usually by small, non-relevant modifications (e.g., a change in the type of solvent used for retrosynthesis single-step prediction). In order to enhance diversity in such approaches, embodiments of the present invention advantageously introduce class identifiers (e.g., as tokens of macro classes in the inputs) as described herein. As a result, the learned embeddings of a given sample partly codify characteristics of the reactions belonging to that class. With respect to inferences, the macro classes make it possible to steer the model towards different kinds of disconnection strategies. According to this approach, substantial improvements in the diversity of predictions is achieved.

While the use of excessively specific groupings can decrease the model performances in terms of valid, proposed set of precursors, the use of chemically relevant policies to construct smaller macro groups allows for the ability to recover quality predictions without loss of diversity. In this respect, embodiments of the present invention recognize that it is advantageous to rely on classes relating to one or more of the following categories of chemical reactions: heteroatom alkylation & arylation, acylation, c—c bond forming, aromatic heterocycle formation, deprotection, protection, reduction, oxidation, functional group interconversion, functional group addition, and resolution. In addition, embodiments of the present invention further recognize that an additional class may encompass miscellaneous (e.g., unrecognized) chemical reactions, so as to allow systematic categorizations. A general retrosynthesis algorithm may advantageously comprise each of the above classes. In an embodiment, the number of classes is limited, e.g., to a number less than or equal to 20, to allow statistically relevant inferences to be performed, while avoiding a decrease in the performance of the model in terms of valid proposed sets of precursors.

In an embodiment, a systematic approach may be contemplated, in which the number N of classes considered on inferences is equal to M. In an alternate embodiment, only a subset of the number of N classes may be used for inference purposes. In an embodiment, the subset may be automatically selected based on the test input selected, in which machine learning may again be used to achieve such automatic selection. In an embodiment, a user may select appropriate classes for a given test input.

In embodiments of the present invention, various machine learning models and corresponding cognitive algorithms may be used, starting with feedforward neural networks (e.g., multilayer perceptrons, deep neural networks, and convolutional neural networks). In an embodiment, the machine learning model used is based on a specific type of architecture, involving an encoder-decoder structure (e.g., as part of a sequence-to-sequence architecture), wherein one or more encoders are connected to one or more decoders, as schematically illustrated in FIG. 7C. It should be noted, however, that FIG. 7C depicts a single encoder stack and a single decoder stack, for simplicity purposes only.

In an embodiment, each of the encoders and each of the decoders may involve an attention layer (e.g., so as to enable a multi-head attention mechanism) and a feed-forward neural network, where the encoder stack(s) and decoder stack(s) interoperate so as to perform the desired inferences by predicting probabilities of possible outputs. Such outputs may then be selected based, at least in part, on their likelihood (which reflects the confidence of the model), and the classes respectively associated to the inputs, so as to allow class-dependent inference results to be returned. In other words, attention layers replace recurrent layers as commonly used in known encoder-decoder architectures.

As noted above, the encoding component may actually include a stack of encoders (all identical in structure), while the decoding component may similarly include a stack of a same number of decoders. For example, each encoder's inputs may first flow through a self-attention layer, which helps the encoder to inspect other tokens in the input as it encodes a specific token. The outputs of the self-attention layer are fed to a feed-forward neural network, similar to so-called seq2seq models. Accordingly, the achieved attention mechanism allows global dependencies to be drawn between inputs and various possible outputs, taking into account the different classes. For example, the so-called Transformer network architecture may be used. However, in an alternate embodiment, known recurrence and convolution layers may be used in place of attention layers. However, using attention layers allows for significant improvements in terms of both parallelization.

Beyond retrosynthetic analyses, embodiments of the present invention can advantageously be used for computer-aided design (e.g., to identify given sets of parts composing a given product, according to a given version thereof), computer-aided engineering (e.g., to identify given sets of parts used to manufacture a given product according to given processes), as well as defect or failure predictions, amongst other examples.

In any application, embodiments of the present invention make it possible to reduce confidence bias in machine learning-based inferences while increasing the variability in inference options such that solutions belonging to different regions of the training dataset may be correctly identified, irrespective of the overall confidence.

As noted earlier, inferences may not necessarily be performed for all of the classes (e.g., when N<M). For example, a smaller number of N possible class identifiers may automatically be selected using a cognitive model specifically trained to that aim. In an embodiment, a user may select relevant classes for a given test input. It should be further noted that embodiments of the present invention may be practiced utilizing several test inputs. For example, a test dataset may initially be accessed at step 320 in accordance with FIG. 3, which includes several test inputs, which can each be processed, as described above, be it successively or in parallel.

Referring now to FIG. 4, a flowchart diagram of a method to obtain a cognitive model for use in generating inferences (e.g., in accordance with FIG. 3) in accordance with at least one embodiment of the present invention is depicted. During training to obtain the cognitive model, examples are classified (i.e., arranged into classes or categories) based on each of the respective different class identifiers aggregated with an example input(s). It should be noted that the same class identifiers aggregated with the example input(s) should be used to perform inferences on the test input(s). The example inputs used during the training will preferably involve duplicates (e.g., same reaction products), which, depending on the class assigned thereto, may yield different example outputs (e.g., different sets of precursors as used in different types of chemical reactions). This, in turn, will allow more relevant class-dependent inferences to be performed.

At step 410, a training set is accessed. In an embodiment, the training set includes suitably prepared examples, where each example associates an example input data structure with a respective example output. As noted earlier, each example input data structure if formed by aggregating an example input with a respective different one of the N class identifiers.

At step 420, each example input data structure and each respective example output is tokenized.

At step 430, embedding is performed (e.g., via a feature extraction algorithm), where N set of features are extracted from each of the tokenized versions of the example input data structures and each of respective tokenized versions of the example outputs. In an embodiment, features are extracted as arrays of numbers (e.g., vectors) and thus, the resulting embeddings are vectors or sets of vectors. It should be noted that the features extracted are impacted by the aggregations of the test input with the respective ones of the class identifiers.

In an embodiment, the embedding algorithm may form part of the training algorithm used to train the cognitive model. In an embodiment, embedding is performed separately and/or prior to training the cognitive model (e.g., prior to step 440). In an embodiment, in addition to feature extraction, embedding may further include a feature selection algorithm and/or dimension reduction as known by one of ordinary skill in the art. In an embodiment, other (though related) embedding algorithms may be used, instead of and/or in addition to feature extraction. For example, dimension reduction may be applied in output or as part of the feature extraction algorithm, as known by one of ordinary skill in the art.

At step 440, a cognitive model is trained using the examples associating the example input data structures with the respective example outputs. At step 450, the parameters of the trained cognitive model are stored for use in performing inferences on test data (e.g., in accordance with FIG. 3).

It should be noted that that the terms “cognitive algorithm”, “cognitive model”, “machine learning model”, or the like, are often used interchangeably. However, for clarification purposes, the underlying training process may be described as follows: A machine learning model (or a cognitive model) is generated by a cognitive algorithm, which learns its parameter(s) from the examples provided during a training phase, so as to arrive at a trained model. Thus, a distinction can be made between the cognitive algorithm used to train the model and the model itself (i.e., the object that is eventually obtained upon completion of the training, and which can be used for inference purposes).

Whereas the inferences performed at step 360 are based on N sets of features extracted at step 350 from the N input data structures in accordance with FIG. 3, for consistency purposes, the machine learning model used for performing such inferences must also be trained based on features extracted from examples, including features extracted from the example input data structures. In this scenario, each input data structure is first formed by aggregating a corresponding test input with a respective different one of the N identifiers. Then, features of each respective test input data structure are extracted (e.g., to form a feature vector). The cognitive model must be similarly obtained. Accordingly, features extracted from the example data input structures reflect aggregations of the example inputs with the class identifiers, irrespective of associations between the example data structures and the corresponding outputs. However, the training of the machine learning model is based on features extracted from the examples as a whole (i.e., including the example outputs) and therefore takes into account associations between the example data input structures and the corresponding outputs.

In an embodiment, the aggregations may be performed a posteriori. That is, features are first extracted from the inputs and then corresponding vectors are aggregated (i.e., concatenated) with additional numbers (or vectors) representing the class identifiers. That is, one may first extract features (in a machine learning sense) and then form aggregations. In this case, computations are performed based on input data structures each formed by aggregating features (vectors) extracted from the test input with features (vectors) extracted from respective different ones of the N class identifiers.

Referring now to FIG. 5, a method for preparing a training set of suitable examples for training a machine learning model that can subsequently be used to perform class-dependent inferences associated with chemical retrosynthetic analysis in accordance with at least one embodiment of the present invention is depicted. At step 510, an example datafile or record is accessed, which includes information as to a given input and a corresponding output. For example, as depicted in FIG. 6A, the example may concern a given chemical reaction, including a given chemical product (input) and given precursors (outputs). As further depicted in FIG. 6A, 2-(4-Cyclopropanecarbonyl-phenyl)-2-methyl-propionic acid is the input and 2-(4-Cyclopropanecarbonyl-phenyl)-2-methyl-propionitrile, ethanol, and sodium hydroxide are the outputs. Such inputs and outputs have the following string representation with the SMILE system syntax (depicted in FIG. 6B):

Product:CC(C)(C(═O)O)c1ccc(C(═O)C2CC2)cc1

Precursors:CC(C)(C #N)c1ccc(C(═O)C2CC2)cc1.CCO.O[Na].

At step 520, the above listed example is automatically classified, for example, using an automated process, which appropriately identifies a functional group interconversion corresponding to class identifier 9 (depicted in FIG. 6C).

At step 530, class identifier 9 is aggregated with the above listed example input to form an input data structure (depicted in FIG. 6D).

At step 540, the input data structure (generated in step 530) is associated with the example output to form a suitable example datum (depicted in FIG. 6E) that can be used for training purposes. It should be appreciated that the aggregation of a class identifier (e.g., class identifier 9) may also be formed after the association, contrary to FIGS. 6D and 6E.

At step 550, the obtained example datum (depicted in FIG. 6E) is stored in a training dataset. It should be appreciated that steps 510-550 may be repeated until a sufficient sized training dataset is achieved.

The final training dataset can be used to train the machine learning model (e.g., in accordance with FIG. 4) for subsequent use to perform class-dependent machine learning based inferences associated with chemical retrosynthetic analysis. Referring again now to FIG. 4, at step 410, the final training set of suitably prepared examples is accessed. At step 420, the example input data structures and outputs of each example are tokenized (depicted in FIG. 6F) for an example input data structure. This yields n tokens (depicted in FIG. 6G), which can then be used for embedding purposes at step 430. The embedding process (or feature extraction) is based on the extracted tokens of each example, yielding sets of vectors that are fed to train the cognitive model at step 440. Upon completion of the training, parameters of the cognitive model obtained are stored at step 450.

The parameters of the cognitive model obtained and stored at step 450 can be used to perform class-dependent machine learning based inferences associated with chemical retrosynthetic analysis (e.g., in accordance with FIG. 3). Referring again now to FIG. 3, a user selection of a given test input is received at step 310 and accessed at 320, together with N class identifiers. The number of N class identifiers may be provided (or selected) by the user or automatically inferred, as noted earlier.

At step 330, N input data structures are formed by aggregating the test input with respective different ones of the N identifiers accessed. At step 340, each of the input data structures are tokenized based on the feature extraction (embedding) process at step 350 (as depicted in FIGS. 7A and 7B). At step 360, the cognitive model trained in accordance with FIG. 4 is loaded to perform inferences for each input data structure (e.g. as depicted in FIG. 7C, which assumes an encoder-decoder model implementing an attention mechanism, as discussed earlier). At step 370, class-dependent inference results are returned to the user. At step 380, the user may utilize the inference results returned (e.g., select precursors returned for a given class and make them react according to the corresponding chemical reaction).

One of ordinary skill in the art will appreciate that a similar pipeline may, for instance, be used in a computer-aided engineering system, to identify parts to be fabricated according to a given process to obtain a given product.

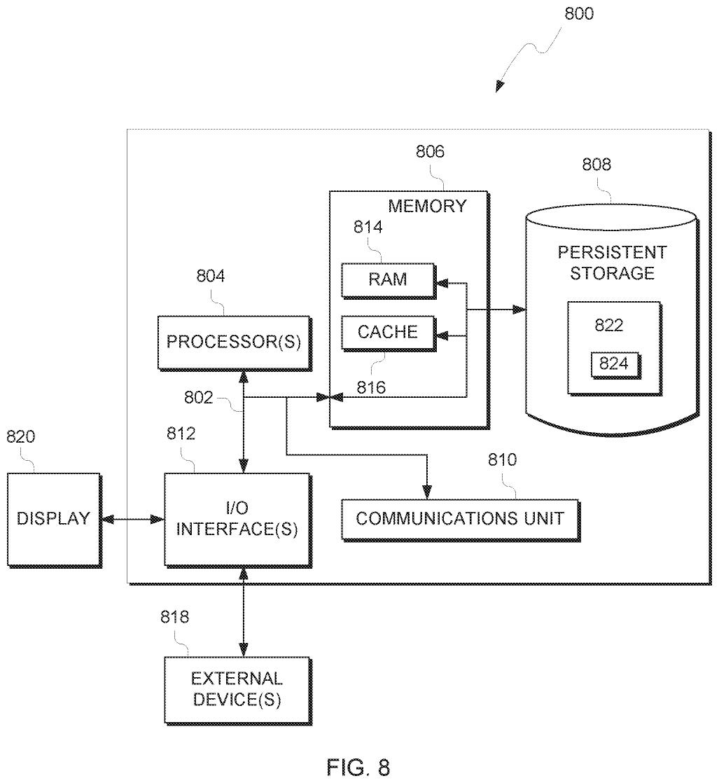

Referring now to FIG. 8 a computing device 800 of cloud computing node 10 (depicted in FIG. 1) in accordance with at least one embodiment of present invention is disclosed. It should be appreciated that FIG. 8 provides only an illustration of one implementation and does not imply any limitations with regard to the environments in which different embodiments may be implemented. Many modifications to the depicted environment may be made.

As depicted in FIG. 8, computing device 800 of cloud computing node 10 includes communications fabric 802, which provides communications between computer processor(s) 804, memory 806, persistent storage 808, communications unit 810, and input/output (I/O) interface(s) 812. Communications fabric 802 can be implemented with any architecture designed for passing data and/or control information between processors (such as microprocessors, communications and network processors, etc.), system memory, peripheral devices, and any other hardware components within a system. For example, communications fabric 802 can be implemented with one or more buses.

Memory 806 and persistent storage 808 are computer-readable storage media. In this embodiment, memory 806 includes random access memory (RAM) 814 and cache memory 816. In general, memory 806 can include any suitable volatile or non-volatile computer-readable storage media.

Program/utility 822, having one or more program modules 824 are stored in persistent storage 808 for execution and/or access by one or more of the respective computer processors 804 via one or more memories of memory 806. Program modules 824 generally carry out the functions and/or methodologies of embodiments of the present invention as described herein. In an embodiment, persistent storage 808 includes a magnetic hard disk drive. Alternatively, or in addition to a magnetic hard disk drive, persistent storage 808 can include a solid state hard drive, a semiconductor storage device, read-only memory (ROM), erasable programmable read-only memory (EPROM), flash memory, or any other computer-readable storage media that is capable of storing program instructions or digital information.

The media used by persistent storage 808 may also be removable. For example, a removable hard drive may be used for persistent storage 808. Other examples include optical and magnetic disks, thumb drives, and smart cards that are inserted into a drive for transfer onto another computer-readable storage medium that is also part of persistent storage 808.

Communications unit 810, in these examples, provides for communications with other data processing systems or devices, including resources of cloud computing environment 50. In these examples, communications unit 810 includes one or more network interface cards. Communications unit 810 may provide communications through the use of either or both physical and wireless communications links. Program modules 824 may be downloaded to persistent storage 808 through communications unit 810.

I/O interface(s) 812 allows for input and output of data with other devices that may be connected to computing device 800. For example, I/O interface 812 may provide a connection to external devices 818 such as a keyboard, keypad, a touch screen, and/or some other suitable input device. External devices 818 can also include portable computer-readable storage media such as, for example, thumb drives, portable optical or magnetic disks, and memory cards. Software and data used to practice embodiments of the present invention, e.g., program modules 824, can be stored on such portable computer-readable storage media and can be loaded onto persistent storage 808 via I/O interface(s) 812. I/O interface(s) 812 also connect to a display 820.

Display 820 provides a mechanism to display data to a user and may be, for example, a computer monitor, or a television screen.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

What is claimed is:

1. A computer-implemented method of performing class-dependent, machine learning based inferences, the method comprising:
accessing a test input and N class identifiers, wherein each class identifier of the N class identifiers identifies a respective class among M possible classes;
forming N test input data structures, wherein each test input data structure of the N test input data structures is formed by aggregating the test input with a different one of the N class identifiers;
generating an inference for each of the N test input data structures using a machine learning model that is trained using examples associating example input data structures with respective example outputs, wherein each respective example input data structure is formed by aggregating an example input with a different one of the N class identifiers; and
returning a class-dependent inference result for each respective test input data structure based on the inference generated for each respective test input data structure.

2. The computer-implemented method of claim 1, further including, prior to accessing the test input:
accessing a training set including the examples associating the example input data structures with the respective example outputs; and
training the machine learning model according to the examples.

3. The computer-implemented method of claim 1, wherein:
inferences are generated based on N sets of features extracted from the N input data structures, respectively; and
the machine learning model is a model trained based on features extracted from the example input data structures.

4. The computer-implemented method of claim 3, wherein:
each of the N test input data structures is formed by concatenating a string representing the test input with a string representing the different one of the N identifiers; and
each of the example input data structures used to train the machine learning model are formed by concatenating a string representing the example input with the string representing the different one of the N class identifiers.

5. The computer-implemented method of claim 4, wherein:
the N sets of features are extracted from tokenized versions of the N input data structures;
the machine learning model is trained based on features extracted from tokenized versions of the example input data structures; and
each of the tokenized versions of the N input data structures and the tokenized versions of the example input data structures are obtained by applying a same tokenization algorithm.

6. The computer-implemented method of claim 1, wherein:
the machine learning model includes an encoder-decoder structure, including one or more encoders connected to one or more decoders, wherein each of the encoders and each of the decoders involves an attention layer and a feed-forward neural network, interoperating so as to generate the inference for each of the N test in put data structures by predicting probabilities of possible outputs.

7. The computer-implemented method of claim 5, wherein:
the strings representing the test input, the example inputs, and the class identifiers are strings obtained according to a same set of syntactic rules; and
the tokenization algorithm is devised in accordance with the syntactic rules.

8. The computer-implemented method of claim 7, further comprising:
generating respective tokens from the strings representing the N class identifier based on the tokenization algorithm.

9. The computer-implemented method of claim 7, wherein:
the strings representing the test input data structures and the strings representing the example data input structures are ASCII strings specifying structures of chemical species corresponding to chemical reaction products; and
each the example outputs used to train the machine learning model are ASCII strings formed by aggregating specifications of structures of two or more precursors of the chemical reaction products.

10. The computer-implemented method of claim 9, wherein:
the ASCII strings are formulated according to a simplified molecular-input line-entry system.

11. The computer-implemented method of claim 1, wherein:
the M possible classes include one or more of the following categories of chemical reactions: heteroatom alkylation and arylation, acylation, c—c bond forming, aromatic heterocycle formation, deprotection, protection, reduction, oxidation, functional group interconversion, functional group addition, and resolution; and
a miscellaneous class for unrecognized chemical reactions.

12. The computer-implemented method 11, wherein:
the M possible classes include classes pertaining to each of the chemical reactions.

13. The computer-implemented method of claim 1, wherein M≥N≥2.

14. The computer-implemented method of claim 1, wherein N=M.

15. The computer-implemented method of claim 1, further comprising:
automatically selecting the n class identifier based on the accessed test input, wherein N<M.

16. The computer-implemented method of claim 1, further comprising, prior to accessing the test input and the N class identifiers:
receiving a user selection of the test input and the N class identifiers.

17. A computer-implemented method of machine learning based retrosynthesis planning, the method comprising:
accessing a test input and N class identifiers, wherein the test input is a string specifying a structure of a chemical species corresponding to a chemical reaction product, and wherein each class identifier of the N class identifiers is a string identifying a respective class among M possible classes of chemical reactions;
forming N test input data structures, wherein each test input data structure of the N test input data structures is formed by concatenating the test input with a different one of the N class identifiers;
generating an inference for each of the N test input data structures using a machine learning model that is trained using examples associating example input data structures with respective example outputs, wherein each respective example input data structure is formed by concatenating an example input with a different one of the N class identifiers, each respective input data structure is a string specifying structures of chemical species corresponding to chemical reaction products, and each respective example output is a string formed by aggregating specifications of structures of two or more precursors of the chemical reaction products; and
returning a class-dependent inference result for each respective test input data structure based on the inference generated for each respective test input data structure.

18. The computer-implemented method of claim 17, further comprising:
performing a chemical reaction according to the class-dependent inference results returned.

19. The computer-implemented method of claim 17, wherein:
the strings representing the test input, the example inputs, the example outputs, and the N class identifiers are strings formed according to a same set of syntactic rules; and
the same set of syntactic rules are in accordance with a simplified molecular-input line-entry system.

20. A computer system for performing class-dependent, machine learning based inferences, the computer system comprising:
one or more computer processors,
one or more computer readable storage media, and
program instructions stored on the computer readable storage media for execution by at least one of the one or more processors, the program instructions including instructions to:
access a test input and N class identifiers, wherein each class identifier of the N class identifiers identifies a respective class among M possible classes;
form N test input data structures, wherein each test input data structure of the N test input data structures is formed by aggregating the test input with a different one of the N class identifiers;
generate an inference for each of the N test input data structures using a machine learning model that is trained using examples associating example input data structures with respective example outputs, wherein each respective example input data structure is formed by aggregating an example input with a different one of the N class identifiers; and
return a class-dependent inference result for each respective test input data structure based on the inference generated for each respective test input data structure.
